(19) United States
(12) Patent Application Publication
(10) Pub. No.: US 2020/0352781 A1
(43) Pub. Date: Nov. 12, 2020

(54) METHODS OF USING ELECTRONIC HEAT THERAPY PATCHES

(71) Applicant: Multitech Medical Devices USA LLC, Holtsville, NY (US)

(72) Inventor: Shuh-Tsai Yang, New Taipei City (TW)

(21) Appl. No.: 16/805,978

(22) Filed: Mar. 2, 2020

Related U.S. Application Data
(60) Provisional application No. 62/846,037, filed on May 10, 2019.

(51) Int. Cl. A61F 7/02; A61F 7/00

(52) U.S. Cl. CPC A61F 7/02; A61F 2007/0093; A61F 2007/0226; A61F 7/007

(57) ABSTRACT

A patch system includes a patch applied to the body using an adhesive pad. The adhesive pad has a plurality of discrete sections of backing that are independently removed from the adhesive pad during application of the patch to a patient's skin or outerwear.

### BACKGROUND

The use of heat to treat muscle and joint pain is well established. Heat therapy using heating pads increases blood circulation and elevates tension in joints and muscles to ease pain. Heating pads have been in existence for over 100 years. Common heat patches use a chemical exothermic reaction that is activated by air when removed from a sealed package. Other types include microwaveable gel packs placed in a holder. These devices enable heat therapy to be mobile instead of using a heating pad that is plugged into a stationary power source. Both the chemical type and the microwaveable type of thermal patches have a temperature change with time. For example, the chemical patch takes several minutes to activate and then for several hours increases in temperature and then for several hours decreases in temperature. The microwaveable type starts hot then decreases in temperature over time. Both the chemical type and the microwaveable type do not have accurate and consistent temperature control.

Another type of heat patch is an electronically-heated heat therapy patch. While the heat distribution with the electronic heat therapy patch does not have the same peaks and valleys as the chemical patches, it does offer a more consistent and even heat flow distribution which is better for the healing process. There are several heat patches using low voltage, as with a USB cable, that wrap around a body part using Velcro® to attach to the fabric or material of the wrap. Wraps are bulky, uncomfortable, and subject to shifting when a small area is to be treated. Using an adhesive has the advantage of keeping the patch in place with no accessories; however, adhesives are not reusable and limit the use to only one time.

### SUMMARY

In accordance with an aspect of the present disclosure, a method of using a multiple use electronically heated patch is provided and includes removing a flexible sheet of an adhesive pad from an adhesive-coated surface of the adhesive pad; attaching an electronically heated patch to the adhesive-coated surface of the adhesive pad; and attaching the adhesive-coated surface of the adhesive pad to a surface of a patient, thereby coupling the electronically heated patch to the patient.

In aspects, removing the flexible sheet may include removing the flexible sheet from a central region of the adhesive-coated surface.

In aspects, the method may further include removing first and second flexible pieces of the adhesive pad from first and second end portions of the adhesive-coated surface, respectively.

In aspects, attaching the adhesive-coated surface may include attaching the first and second end portions of the adhesive-coated surface to the surface of the patient.

In aspects, the first and second flexible pieces may be removed from the first and second end portions of the adhesive-coated surface after the flexible sheet is removed from the central region of the adhesive-coated surface.

In aspects, the electronically heated patch may be attached to the central region of the adhesive-coated surface before the first and second flexible pieces are removed from the first and second end portions of the adhesive-coated surface.

In aspects, removing the flexible sheet from the central region of the adhesive-coated surface may include peeling back a lateral side of the flexible sheet that overlaps with the first flexible piece.

In aspects, removing the first flexible piece may include grasping a tab of the first flexible piece; and peeling back the first flexible piece from the first end portion of the adhesive-coated surface.

In aspects, the electronically heated patch may include a connector configured to couple to a source of power. The connector may be disposed outside of an outer periphery of the adhesive pad upon attaching the electronically heated patch to the adhesive-coated surface of the adhesive pad.

In aspects, attaching the electronically heated patch may include detachably adhering a majority of the electronically heated patch to a central region of the adhesive-coated surface while leaving an end portion of the electronically heated patch disposed outside of an outer periphery of the adhesive pad.

In aspects, removing the flexible sheet may include peeling the flexible sheet from a central region of the adhesive-coated surface.

In aspects, the method may further include electromechanically coupling the electronically heated patch to a source of power.

In aspects, electromechanically coupling the electronically heated patch to a source of power may include plugging a cable connector to a power connector of the electronically heated patch.

In accordance with another aspect of the present disclosure, a multiple use electronically heated patch system is provided and includes an electronically heated patch and an adhesive pad. The patch includes a first flexible layer and a heater coupled to the flexible layer for heating the flexible layer. The adhesive pad includes a second flexible layer having adhesive disposed on a first side of the second flexible layer, and a peelable flexible sheet covering and detachably attached to the first side of the second flexible layer. The first flexible layer is configured to adhere to the first side of the second flexible layer.

In aspects, the adhesive pad may further include peelable first and second flexible pieces covering and detachably attached to respective first and second end portions of the first side of the second flexible layer. The first and second end portions of the first side of the second flexible layer may be configured to adhere to skin of a patient when the flexible sheet and the first and second flexible pieces are detached from the second flexible layer.

In aspects, the flexible sheet, the first flexible piece, and the second flexible piece may be independently detachable from the second flexible layer.

In aspects, the first flexible layer may have a shape and size approximating a shape and size of the central region of the first side of the second flexible layer, such that upon coupling the first flexible layer to the second flexible layer, the first and second end portions of the second flexible layer protrude outwardly from first and second lateral sides of the first flexible layer.

In aspects, the flexible sheet may have first and second end portions that overlap with the respective first and second flexible pieces.

In aspects, each of the first and second flexible pieces may have a tab disposed underneath the respective first and second end portions of the flexible sheet. The tab of each of the first and second flexible pieces may be configured to be grasped by a hand to peel the first and second flexible pieces from the second flexible layer.

In aspects, the electronically heated patch may further include a control circuit coupled to the first flexible layer, and a USB power connector in electrical connection with the control circuit for supplying power.

In aspects, the electronically heated patch may further include an integrated circuit within the control circuit and configured to control the temperature of the heater.

Further details and aspects of exemplary embodiments of the present disclosure are described in more detail below with reference to the appended figures.

As used herein, the terms parallel and perpendicular are understood to include relative configurations that are substantially parallel and substantially perpendicular up to about + or −10 degrees from true parallel and true perpendicular.

### BRIEF DESCRIPTION OF THE DRAWINGS

Embodiments of the present disclosure are described herein with reference to the accompanying drawings, wherein:

FIG. 1 is a top view of an exemplary embodiment of an electric heat patch for applying heat therapy to a user;

FIG. 2 is a bottom view, with a lower fabric layer removed, of the patch of FIG. 1;

FIG. 3 is a side, perspective view illustrating an adhesive pad for use with the patch of FIG. 1;

FIGS. 4A-4D illustrate a sequence of using the patch and adhesive pad of FIGS. 1-3;

FIG. 5A illustrates an application of the patch of FIG. 1 to a knee of a patient;

FIG. 5B illustrates an application of the patch of FIG. 1 to an upper back of a patient;

FIG. 5C illustrates an application of the patch of FIG. 1 to a lower back of a patient; and

FIG. 6 is a flow chart illustrating an exemplary method of using the patch of FIG. 1.

### DETAILED DESCRIPTION

Embodiments of the presently disclosed electric heat patch systems are described in detail with reference to the drawings, in which like reference numerals designate identical or corresponding elements in each of the several views.

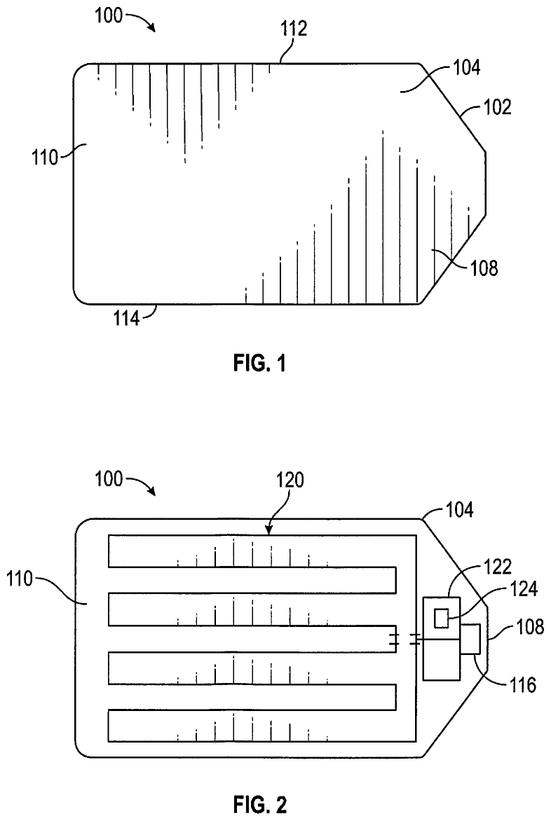

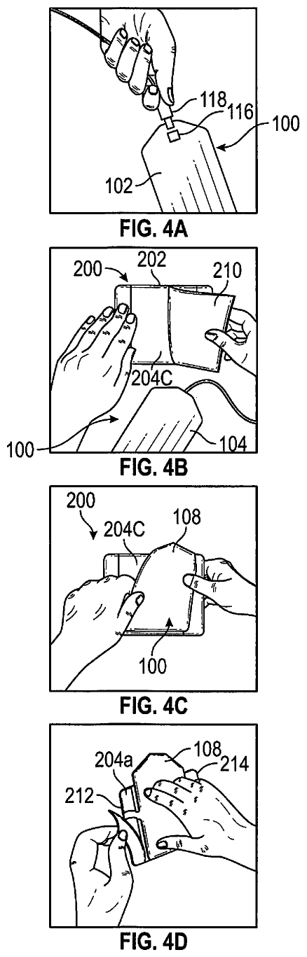

FIGS. 1-3 illustrate an exemplary embodiment of a multi-use electronically heated patch system that includes a heat patch 100 and an adhesive pad 200 for coupling the patch 100 to a patient. The patch 100 includes a flexible lower layer 102 (FIGS. 1 and 4A) and a flexible upper layer 104 bonded or otherwise coupled to the flexible lower layer 102. In aspects, the patch 100 may have only one flexible layer that defines a pocket therein for housing the electronics of the patch 100. The flexible lower and upper layers 102, 104 of the patch 100 may be made of a soft polyester fabric. Other suitable types of fabrics and materials are also contemplated.

The heat patch 100 has opposing first and second end portions 108, 110 and opposing first and second lateral sides 112, 114. The first end portion 108 has an end of a power USB connector 116, such as, for example, a micro USB connector, a mini USB connector, or a standard USB connector, protruding outwardly from the flexible lower layer 102. The connector 116 is configured to receive a connector plug 118 (FIG. 4A) for transferring electricity to the patch 100. The patch 100 includes a heater 120 disposed between the flexible lower and upper layers 102, 104 and adhered thereto by a heat-activated glue (not explicitly shown) having similar properties as hot melt adhesive glue. The heater 120 may include a heater wire alloy fixed in a fabric matrix in a serpentine pattern equally spaced to provide uniform heat to the treated area. The heater 120 and the power USB connector 116 are electrically attached to a printed circuit board 122. The circuit board 122 has a control circuit, such as, for example, an integrated circuit 124 (e.g., a microprocessor uP), and a temperature sensor (not explicitly shown) electrically connected to the printed circuit board 122. The printed circuit board 122 may be made of a rigid fiberglass type or as a flexible type as is known by the industry.

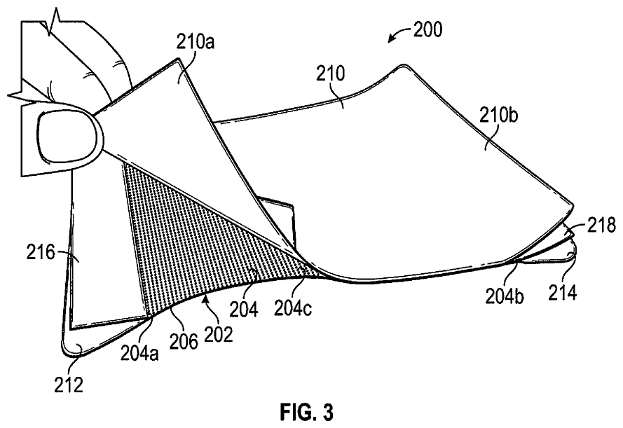

FIG. 3 illustrates the adhesive pad 200 for selectively affixing the patch 100 to a patient's skin or outerwear. The adhesive pad 200 includes a flexible layer 202 having a first side 204 and a second side 206. The first side 204 of the flexible layer 202 has adhesive (not explicitly shown) coated or otherwise disposed thereon, and the second side 206 is fabricated from a non-adhesive fabric material. The first side 204 of the flexible layer 202 has first and second end portions 204a, 204b and a central region 204c disposed between the first and second end portions 204a, 204b. The adhesive coated on the first side 204 of the flexible layer 202 may be a polyurethane adhesive, removable tape (e.g., silicone film), a peelable adhesive gel, or any other suitable adhesive. In some aspects, the adhesive may be a silicone film of the type sold under the name “3M™ Kind Removal Silicone Tape” by 3M company corp. The adhesive is of medical grade and tested for compatibility and long term toxicity.

The adhesive pad 200 includes a backing made of woven fabric, plastic (e.g., PVC, polyethylene or polyurethane), or latex. The backing includes a flexible sheet 210 and first and second flexible pieces 212, 214 each discrete from one another and separately detachable from the adhesive of the flexible layer 202. The flexible sheet 210 of the backing covers and is selectively detachable from the central region 204c of the first side 204 of the flexible layer 202. The flexible sheet 210 has first and second end portions 210a, 210b that overlap, without connecting to, the first and second end portions 204a, 204b of the flexible layer 202.

The first and second flexible pieces 212, 214 of the backing cover and are selectively detachable from the first and second end portions 204a, 204b of the flexible layer 202. As such, the first and second flexible pieces 212, 214 are disposed between the respective first and second end portions 204a, 204b of the flexible layer 202 and the first and second end portions 210a, 210b of the flexible sheet 210 of the backing. Each of the first and second flexible pieces 212, 214 has a bent tab 216, 218 disposed underneath the respective first and second end portions 210a, 210b of the flexible sheet 210. The tabs 216, 218 are configured to be independently grasped by a hand of a user to peel the first and second flexible pieces 212, 214 from the flexible layer 202 of the adhesive pad 200.

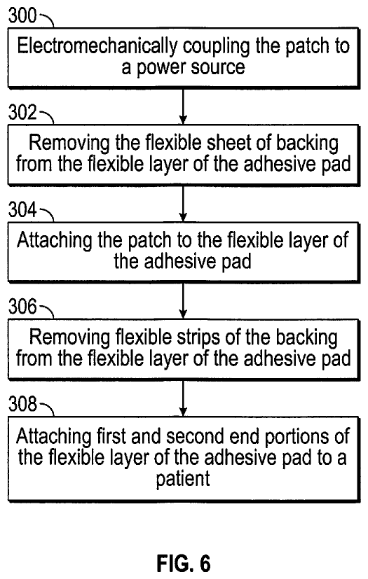

With reference to FIGS. 4A-4D, 5A-5C, and 6, a method of using the electronically heated patch system will now be described. In step 300, the patch 100 is electromechanically coupled to a source of power. In particular, as shown in FIG. 4A, the cable connector 118, which stems from a power source (e.g., a battery or an outlet), is received in the USB connector 116 of the patch 100. In step 302, as shown in FIG. 4B, the flexible sheet 210 of the backing is peeled back from the adhesive-coated central region 204c of the first side 204 of the flexible layer 202 of the adhesive pad 200. In step 304, as shown in FIG. 4C, the lower flexible layer 102 of the patch 100 is positioned over and attached to the adhesive-coated central region 204c of the first side 204 of the adhesive pad 200. Upon attaching the patch 100 to the adhesive pad 200, a majority of the patch 100 is attached to the central region 204c of the adhesive pad 200 while leaving the first end portion 108 of the patch 100 disposed outside of an outer periphery of the adhesive pad 200, such that the USB connector 116 remains accessible.

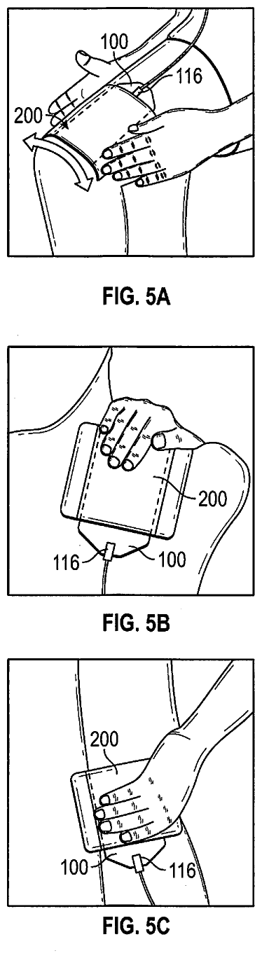

In step 306, as shown in FIG. 4D, the first and second flexible pieces 212, 214 of the backing are peeled back from the adhesive-coated first and second end portions 204a, 204b of the flexible layer 202 of the adhesive pad 200, respectively. The flexible pieces 212, 214 may be removed from the flexible layer 202 by individually grasping the tabs 216, 218 (FIG. 3) of the respective first and second flexible pieces 212, 214 and peeling back the first and second flexible pieces 212, 214 from the flexible layer 202 of the adhesive pad 200. In step 308, with the adhesive of the first and second end portions 204a, 204b of the flexible layer 202 of the adhesive pad 200 now exposed, the first and second end portions 204a, 204b of the adhesive pad 200 are attached to a surface of a patient, thereby coupling the patch 100 to the patient. The patch 100 may be coupled to any suitable portion of a patient's body, such as for example, a knee of the patient (FIG. 5A), an upper back of the patient (FIG. 5B), or a lower back of the patient (FIG. 5C). As seen in FIGS. 5A-5C, the USB connector 116 of the patch 100 faces outward, and therefore remains accessible by the user when the patch is adhered to the patient.

It will be understood that various modifications may be made to the embodiments disclosed herein. Therefore, the above description should not be construed as limiting, but merely as exemplifications of various embodiments. Those skilled in the art will envision other modifications within the scope and spirit of the claims appended thereto.

1. A method of using a multiple use electronically heated patch, comprising:
removing a flexible sheet of an adhesive pad from an adhesive-coated surface of the adhesive pad;
attaching an electronically heated patch to the adhesive-coated surface of the adhesive pad; and
attaching the adhesive-coated surface of the adhesive pad to a surface of a patient, thereby coupling the electronically heated patch to the patient.

2. The method according to claim 1, wherein removing the flexible sheet includes removing the flexible sheet from a central region of the adhesive-coated surface.

3. The method according to claim 2, further comprising removing first and second flexible pieces of the adhesive pad from first and second end portions of the adhesive-coated surface, respectively.

4. The method according to claim 3, wherein attaching the adhesive-coated surface includes attaching the first and second end portions of the adhesive-coated surface to the surface of the patient.

5. The method according to claim 3, wherein the first and second flexible pieces are removed from the first and second end portions of the adhesive-coated surface after the flexible sheet is removed from the central region of the adhesive-coated surface.

6. The method according to claim 3, wherein the electronically heated patch is attached to the central region of the adhesive-coated surface before the first and second flexible pieces are removed from the first and second end portions of the adhesive-coated surface.

7. The method according to claim 3, wherein removing the flexible sheet from the central region of the adhesive-coated surface includes peeling back a lateral side of the flexible sheet that overlaps with the first flexible piece.

8. The method according to claim 7, wherein removing the first flexible piece includes:
grasping a tab of the first flexible piece; and
peeling back the first flexible piece from the first end portion of the adhesive-coated surface.

9. The method according to claim 1, wherein the electronically heated patch includes a connector configured to couple to a source of power, the connector being disposed outside of an outer periphery of the adhesive pad upon attaching the electronically heated patch to the adhesive-coated surface of the adhesive pad.

10. The method according to claim 1, wherein attaching the electronically heated patch includes detachably adhering a majority of the electronically heated patch to a central region of the adhesive-coated surface while leaving an end portion of the electronically heated patch disposed outside of an outer periphery of the adhesive pad.

11. The method according to claim 1, wherein removing the flexible sheet includes peeling the flexible sheet from a central region of the adhesive-coated surface.

12. The method according to claim 1, further comprising electromechanically coupling the electronically heated patch to a source of power.

13. The method according to claim 12, wherein electromechanically coupling the electronically heated patch to a source of power includes plugging a cable connector to a power connector of the electronically heated patch.

14. A multiple use electronically heated patch system, comprising:
an electronically heated patch including:
a first flexible layer; and
a heater coupled to the flexible layer for heating the flexible layer; and
an adhesive pad including:
a second flexible layer having adhesive disposed on a first side of the second flexible layer; and
a peelable flexible sheet covering and detachably attached to the first side of the second flexible layer, wherein the first flexible layer is configured to adhere to the first side of the second flexible layer.

15. The multiple use electronically heated patch system according to claim 14, wherein the adhesive pad further includes peelable first and second flexible pieces covering and detachably attached to respective first and second end portions of the first side of the second flexible layer, the first and second end portions of the first side of the second flexible layer being configured to adhere to skin of a patient when the flexible sheet and the first and second flexible pieces are detached from the second flexible layer.

16. The multiple use electronically heated patch system according to claim 15, wherein the flexible sheet, the first flexible piece, and the second flexible piece are independently detachable from the second flexible layer.

17. The multiple use electronically heated patch system according to claim 15, wherein the first flexible layer has a shape and size approximating a shape and size of the central region of the first side of the second flexible layer, such that upon coupling the first flexible layer to the second flexible layer, the first and second end portions of the second flexible layer protrude outwardly from first and second lateral sides of the first flexible layer.

18. The multiple use electronically heated patch system according to claim 15, wherein the flexible sheet has first and second end portions that overlap with the respective first and second flexible pieces.

19. The multiple use electronically heated patch system according to claim 18, wherein each of the first and second flexible pieces has a tab disposed underneath the respective first and second end portions of the flexible sheet, the tab of each of the first and second flexible pieces is configured to be grasped by a hand to peel the first and second flexible pieces from the second flexible layer.

20. The multiple use electronically heated patch system according to claim 14, wherein the electronically heated patch further includes:
a control circuit coupled to the first flexible layer; and
a USB power connector in electrical connection with the control circuit for supplying power.
